(19) United States
(12) Patent Application Publication
(10) Pub. No.: US 2019/0096528 A1
(43) Pub. Date: Mar. 28, 2019

(54) MEDICAL EVALUATION SYSTEM

(71) Applicant: ZENXMED INNOVATIONS INC., Edmonton (CA)

(72) Inventors: Colm J. Murphy, Edmonton (CA); Christina E. Lang, Edmonton (CA); Shaun Scholtz, Edmonton (CA); Chris Ryan, Edmonton (CA)

(73) Assignee: ZENXMED INNOVATIONS INC., Edmonton (CA)

(21) Appl. No.: 16/200,160

(22) Filed: Nov. 26, 2018

Related U.S. Application Data
(63) Continuation-in-part of application No. PCT/IB2018/052008, filed on Mar. 23, 2018.
(60) Provisional application No. 62/476,568, filed on Mar. 24, 2017.

(51) Int. Cl. G16H 50/20; G06F 3/0482; G06F 3/0485; G16H 70/00; G06F 16/248

(52) U.S. Cl. CPC G16H 50/20; G06F 3/0482; G06F 2203/04803; G16H 70/00; G06F 16/248; G06F 3/0485

(57) ABSTRACT

In various embodiments, a method may comprise receiving, by a processor, an inquiry in a search bar; displaying, by the processor, a graphical user interface (GUI) inquiry map comprising a plurality of analysis stages related to the inquiry on a display screen, wherein the plurality of analysis stages are displayed proximate to related analysis stages in a logical order, and wherein each analysis stage comprises at least one node linked to a node in an adjacent stage; receiving, by the processor, a first selection of a first node in a first analysis stage of the at least one background factor; and displaying, by the processor, a second analysis stage related to the first selection of the first node proximate the first analysis stage.

### CROSS REFERENCE TO RELATED APPLICATIONS

This application is a Continuation-In-Part of, and claims priority to and the benefit of, PCT Application No. PCT/IB2018/052008, filed on Mar. 23, 2018 and entitled “MEDICAL EVALUATION SYSTEM,” which claims priority to U.S. provisional patent application No. 62/476,568, filed on Mar. 24, 2017 and entitled “MEDICAL EVALUATION SYSTEM,” both of which are incorporated by reference herein in their entireties.

### FIELD

The present disclosure generally relates to a system for providing medical evaluation and information.

### BACKGROUND

The medical field is constantly developing new technologies for disease diagnosis, treatment, prevention, evaluation, and the like. Such rapid development makes it nearly impossible for physicians and other medical professionals to stay completely informed on the current state of medical care on every topic which they may encounter in practice. Additionally, each patient has unique characteristics and medical needs, and therefore, physicians must make medical decisions taking into consideration the uniqueness of each patient (medications, comorbidities, genotypes, financial coverage, geographic origin, illness level) to determine the best course of action. Implementing the most current medical information by physicians and other medical professionals in treating patients is therefore difficult with the abundance of technological advancements and unique patient scenarios.

### SUMMARY

Systems and methods are disclosed relating to a medical evaluation system. In various embodiments, the system may be configured to allow a user (e.g., a medical professional) to input various aspects and characteristics of a situation and/or patient into the system, so the system provides follow-up inquiries to understand the situation/patient, in order to make a medical evaluation and/or give the user guidance on next steps. The system comprises up-to-date medical information and developments, and therefore helps the user be aware of available medical resources that can be tailored to a particular situation. Additionally, the system may allow a user to skip aspects of a medical evaluation that the user already knows, and therefore, maximizes the efficiency of the evaluation process. The system displays the relevant information on a graphical user interface (GUI) in a logical sequence so the user can easily backtrack or skip forward to analysis stages within the evaluation process he or she wishes to review.

In various embodiments, a method (which may be implemented by a system configured to perform operations) may comprise, or a system comprising a processor may perform operations comprising, receiving, by a processor, an inquiry in a search feature; displaying, by the processor, search results on a display screen in response to the inquiry; displaying, by the processor, at least one analysis stage on a continuous GUI on a display screen, wherein the at least one analysis stage comprises a plurality of considerations; receiving, by the processor, a selection of a consideration of the plurality of considerations; determining, by the processor, a recommendation based on the selection of the consideration; and displaying, by the processor, the recommendation on the continuous GUI proximate to the plurality of considerations.

In various embodiments, displaying at least one analysis stage may comprise displaying, by the processor, at least one background factor for selection relating to a patient on a continuous GUI on the display screen, wherein receiving the selection comprises receiving, by the processor, at least one selection of the at least one background factor. In various embodiments, the recommendation is a test recommendation, and wherein the method further comprises receiving, by the processor, a test outcome input associated with the test recommendation, wherein options for the test outcome input are displayed on the continuous GUI proximate to the test recommendation; determining, by the processor, a diagnostic conclusion in response to the receiving the test outcome; and displaying, by the processor, the diagnostic conclusion on the continuous GUI proximate to the recommendation. In various embodiments, the method may further comprise determining, by the processor, a follow-up recommendation or a treatment recommendation in response to the determining the diagnostic conclusion; and displaying, by the processor, at least one of the follow-up recommendation or the treatment recommendation on the continuous GUI proximate to the diagnostic conclusion. In various embodiments, the method may further comprise receiving, by the processor, a tangential consideration related to the patient; determining, by the processor, an adjusted recommendation in response to receiving the tangential consideration; and displaying, by the processor, the adjusted recommendation on the continuous GUI proximate to the tangential consideration.

In various embodiments, the continuous GUI may allow different portions of the continuous GUI to be viewed on the display screen, by the user moving or scrolling to other parts of the continuous GUI. Therefore, the method may further comprise moving, by the processor, a portion of the continuous GUI on the display screen in response to receiving, by the processor, a moving command, wherein at least a portion of the continuous GUI is displayed on the display screen. In various embodiments, the method may further comprise displaying, by the processor, an information button on at least one of the at least one background factor, the test recommendation, the test outcome, the diagnostic conclusion, the follow-up recommendation, or the adjusted recommendation; and displaying, by the processor, information on the continuous GUI in response to a selection of the information button. In various embodiments, the method may further comprise displaying, by the processor, a treatment recommendation based on at least one of the diagnostic conclusion, the tangential consideration, or the at least one background factor, wherein the treatment recommendation is displayed proximate to the diagnostic conclusion. In various embodiments, the method may further comprise determining, by the processor, trial options associated with the diagnostic conclusion based on at least one of the at least one background factor, a treatment recommendation, or the tangential consideration; and displaying, by the processor, the trial options on the continuous GUI.

In various embodiments, a method may comprise receiving, by a processor, an inquiry in a search bar; displaying, by the processor, a graphical user interface (GUI) inquiry map comprising a plurality of analysis stages related to the inquiry on a display screen, wherein the plurality of analysis stages are displayed proximate to related analysis stages in a logical order, and wherein each analysis stage comprises at least one node linked to a node in an adjacent stage; receiving, by the processor, a first selection of a first node in a first analysis stage of the at least one background factor; and displaying, by the processor, a second analysis stage related to the first selection of the first node proximate the first analysis stage.

In various embodiments, the method may further comprise highlighting, by the processor, the first node and a link between the first node and the second analysis stage in response to the receiving the first selection. In various embodiments, the method may further comprise moving, by the processor, a portion of the GUI inquiry map on the display screen in response to receiving, by the processor, a moving command, wherein at least a portion of the GUI inquiry map is displayed on the display screen. In various embodiments, the method may further comprise receiving, by the processor, an analysis stage selection of a selected analysis stage of the plurality of analysis stages, wherein the analysis stage selection allows a user to skip at least one preceding analysis stage preceding the selected analysis stage in the logical order. In various embodiments, the method may further comprise highlighting, by the processor, the preceding analysis stage(s) skipped by the analysis stage selection of the selected analysis stage and at least one preceding link linking the selected analysis stage and the at least one preceding analysis stage. In various embodiments, the method may further comprise displaying, by the processor, a continuous GUI on the display screen comprising information associated with the analysis stage selection, wherein the continuous GUI comprises at least one of factors and considerations relating to a patient to aid in making a final recommendation; receiving, by the processor, at least one selection of the at least one background factor; and/or determining, by the processor, a recommendation based on the at least one selection. In various embodiments, the recommendation is at least one of a diagnostic test or a treatment plan. In various embodiments, the method may further comprise displaying, by the processor, an information button on at least one of an analysis stage of the plurality of analysis stages or the at least one node; and displaying, by the processor, information on the display screen in response to a selection of the information button related to the analysis stage or at least one node associated with the information button.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures. Elements with like element numbering throughout the figures are intended to be the same.

FIG. 1 illustrates an exemplary medical evaluation system, in accordance with various embodiments;

FIGS. 2A-2C illustrate exemplary display screens of a medical evaluation system, in accordance with various embodiments;

FIGS. 3A-3C illustrate exemplary views of a continuous GUI of a medical evaluation system, in accordance with various embodiments;

FIG. 4 illustrates a flowchart depicting an exemplary method analyzing information using a continuous GUI of a medical evaluation system, in accordance with various embodiments;

FIG. 5 illustrates an exemplary GUI inquiry map of a medical evaluation system, in accordance with various embodiments;

FIG. 6 illustrates a flowchart depicting an exemplary method analyzing information using a GUI inquiry map of a medical evaluation system, in accordance with various embodiments;

FIG. 7 illustrates an exemplary summary window in a windowed GUI, in accordance with various embodiments;

FIG. 8A illustrates an exemplary analysis stage in a windowed GUI, in accordance with various embodiments;

FIG. 8B illustrates another exemplary analysis stage in a windowed GUI, in accordance with various embodiments;

FIG. 9 illustrates an exemplary history window in a windowed GUI, in accordance with various embodiments; and

FIG. 10 illustrates a flowchart depicting an exemplary method for accessing information using a windowed GUI of a medical evaluation system, in accordance with various embodiments.

### DETAILED DESCRIPTION

The present disclosure generally relates to regulating transactions by receiving and analyzing transaction identification information. The detailed description of various embodiments herein makes reference to the accompanying drawings, which show the exemplary embodiments by way of illustration. While these exemplary embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

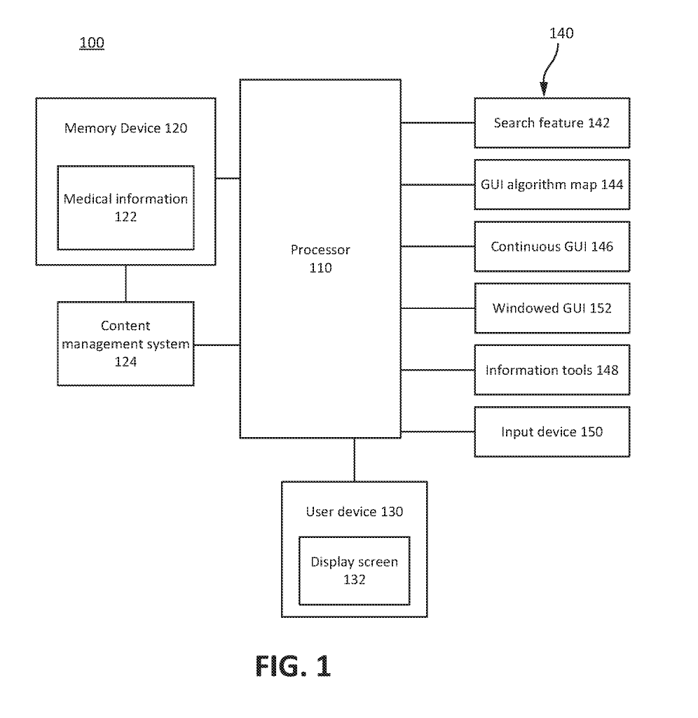

With reference to FIG. 1, an exemplary medical evaluation system 100 is disclosed, in accordance with various embodiments. System 100 may allow a physician to access current information in medicine easily and efficiently. For example, a physician may search a topic of interest using system 100, but the physician may be familiar with the topic. Therefore, the physician may skip general information about the topic using system 100 to get to more pertinent information pertaining to a certain situation. A physician or other medical professional's ability to navigate through information to find what is needed aids in the medical professionals being able to efficiently make medical decisions using the most current information available. System 100 may comprise software integrated with and utilized by a user device 130 (e.g., a personal computer, smartphone, tablet, or the like) having access to a network to allow users (e.g., medical professionals) access and use the information provided by system 100.

In various embodiments, system 100 may comprise a processor 110, a memory device 120, a user device 130 connected to a network, having a display screen 132, and various features which may be part of a graphical user interface (GUI) 140 displayed on display screen 132 of a user device. In various embodiments, processor 110 and/or memory device 120 may be stored on a server separate from, and in communication with, system 100. System 100 may be computer-based and have a network interface.

In various embodiments, processor 110 may comprise hardware and/or software capable of storing data and/or analyzing information. Processor 110 may comprise a server appliance running a suitable server operating system (e.g., MICROSOFT INTERNET INFORMATION SERVICES or, “IIS”) and having database software (e.g., ORACLE) installed thereon. Processor 110 may be in electronic communication with memory device 120 and user device 130 and its display screen 132 via a network. Processor 110 may be configured to process information requests, navigate information based on inputs received by system 100 from a user, and present/display data and information (or cause data and information to be presented and/or displayed) to users of system 100 via display screen 132.

In various embodiments, memory device 120 may comprise hardware and/or software capable of storing data and/or analyzing information. Memory device 120 may comprise a server appliance running a suitable server operating system (e.g., MICROSOFT INTERNET INFORMATION SERVICES or, “IIS”) and having database software (e.g., ORACLE) installed thereon. Memory device 120 may be or may comprise a tangible non-transitory computer-readable memory. Instructions stored on the tangible non-transitory memory may allow and/or instruct processor 110 and system 100 to perform various functions (e.g., navigating and presenting medical information), as described herein. Memory device 120 may store medical information 122 through which processor 110 may navigate in response to search or navigation inputs received by system 100 from a user. Medical information 122 may be displayed on display screen 132 in response to specific inquiries and selections made by a user of system 100.

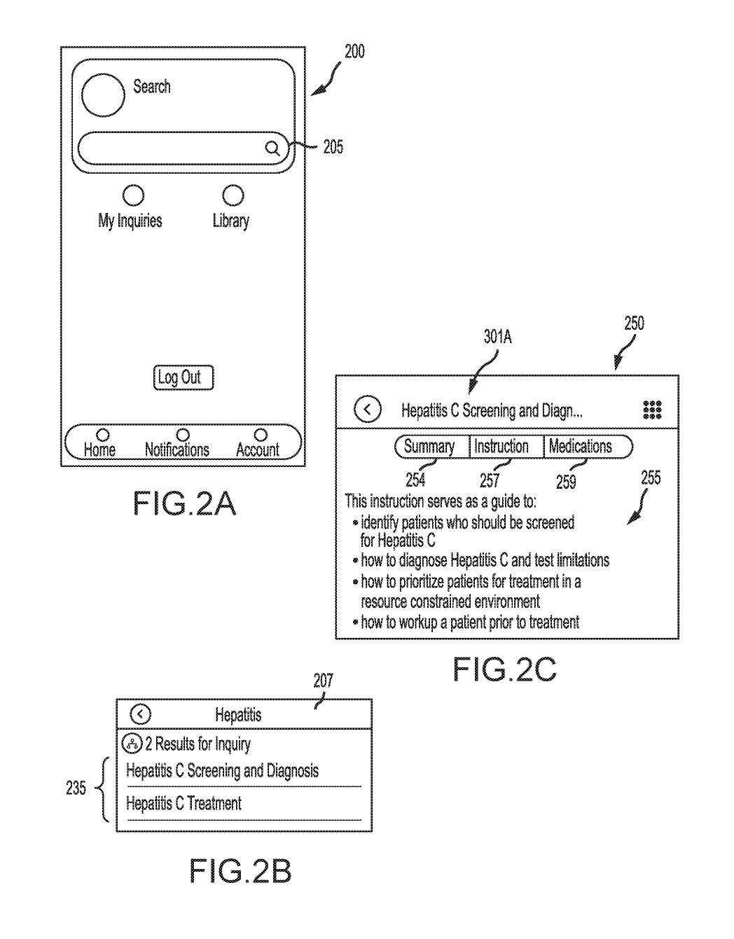

In various embodiments, system 100 may comprise a GUI 140. GUI 140 may take different forms and/or comprise various features, and may be presented on display screen 132 of user device 130. For example, GUI 140 may comprise a search feature 142, information tools 148, and/or an input device 150. With additional reference to FIG. 2A, a user of system 100 may search for medical information by typing an inquiry into search feature 205 (which is an example of search feature 142). Search feature 205 may be comprised in a home screen 200. Input device 150 may be any aspect of GUI 140 which allows a user of system 100 to produce an input signal received by processor 110, which may command processor 110 to perform an operation. For example, input device 150 may be a digital button displayed on display screen 132 (e.g., a touch screen), or a physical button to input information. In various embodiments, input device 150 may also facilitate the transfer of information from a separate device or system into system 100, such that processor 110 may take such outside information into consideration for processing information, with or without user action.

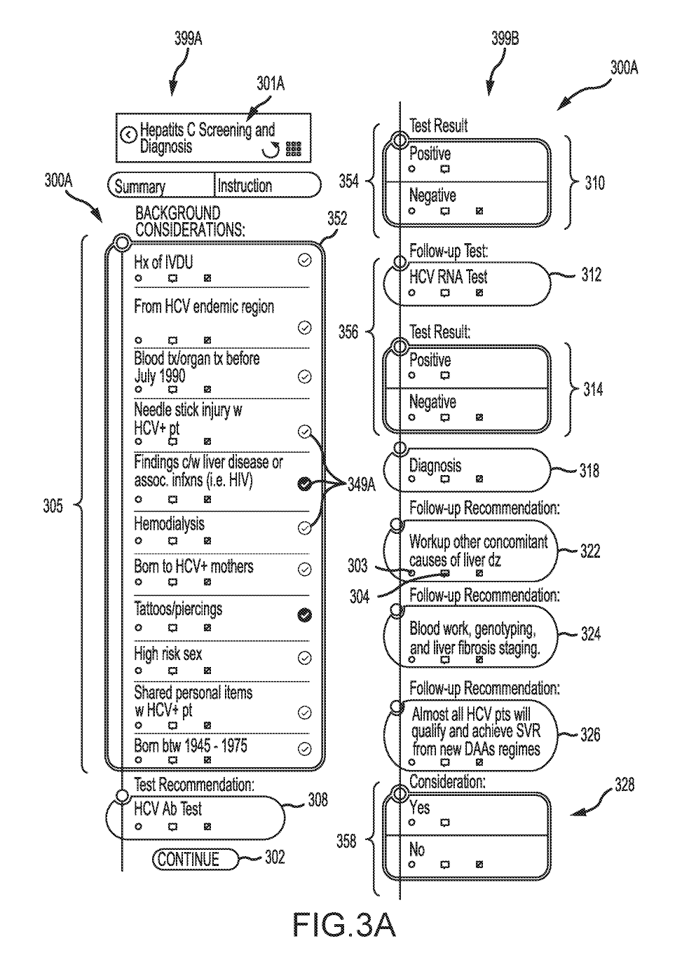

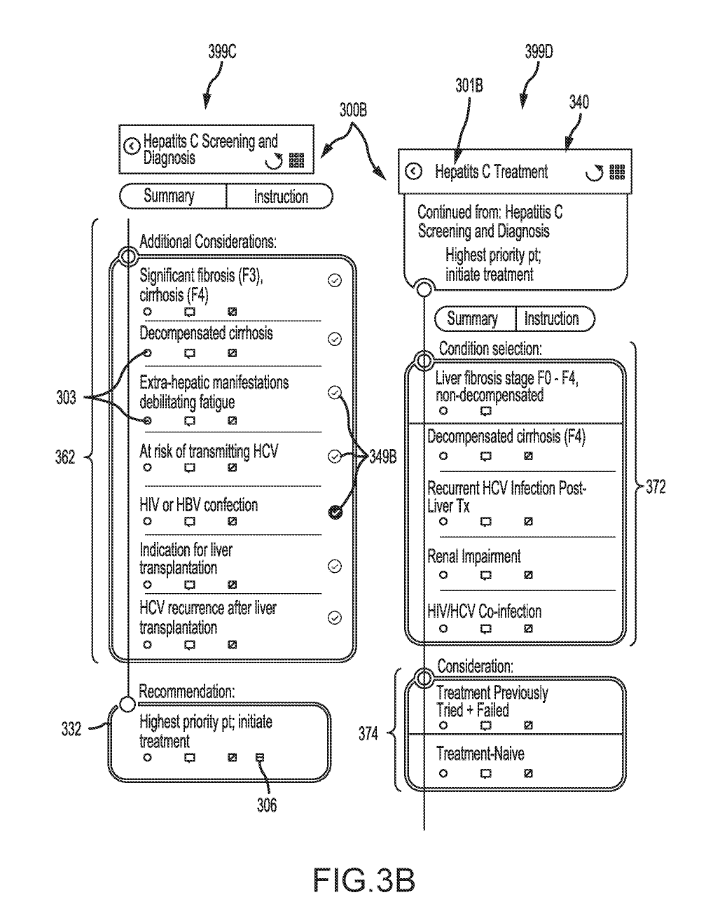

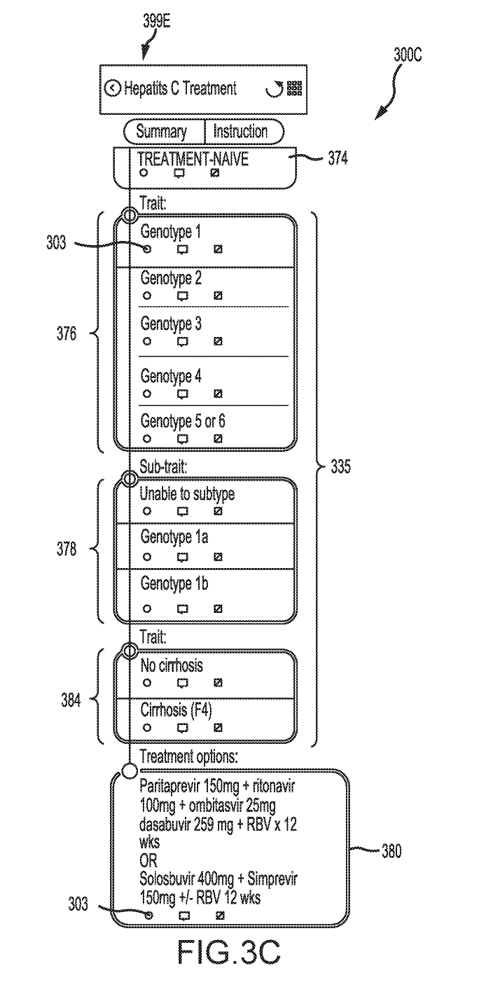

In various embodiments, GUI 140 may take the form of a continuous GUI 146. Continuous GUI 146 may utilize an algorithm for analyzing situations, such as diagnosing a patient with a disease and/or determining an appropriate treatment plan, and with processor 110, may determine and display relevant information on display screen 132 based on user inputs. FIGS. 3A-3C provide various views 300A-300C of a continuous GUI 146, wherein medical information, patient background information, and considerations are displayed by processor 110 on display screen 132 for a user to select if applicable to the present situation and/or patient. Based on the user inputs into continuous GUI 146, processor will determine the next relevant stage of analysis (i.e., an analysis stage) or information to display in continuous GUI 146.

In various embodiments, continuous GUI 146 is a step-by-step process for analyzing information. System 100 is kept up-to-date with the most recent medical data, and therefore, the medical professional using system 100 may be presented with the most recent, relevant information for the subject situation and/or patient. Additionally, continuous GUI 146 provides a continuous view of information in a logical order, wherein each piece of information or consideration for the user is displayed proximate or adjacent to the information on which system 100 based the present piece of information. As used herein, “proximate to” means “close to” and/or adjacent to.”

In various embodiments, system 100 is kept up-to-date by new content being continuously added to system 100 via a content management system 124. In various embodiments, content management system 124 may comprise hardware and/or software capable of receiving and/or storing data, and/or analyzing information. Content management system 124 may comprise a server appliance running a suitable server operating system (e.g., MICROSOFT INTERNET INFORMATION SERVICES or, “IIS”) and having database software (e.g., ORACLE) installed thereon. In various embodiments, content management system 124 may be in communication with memory device 120 and/or processor 110. In various embodiments content management system 124 may be part of memory device 120.

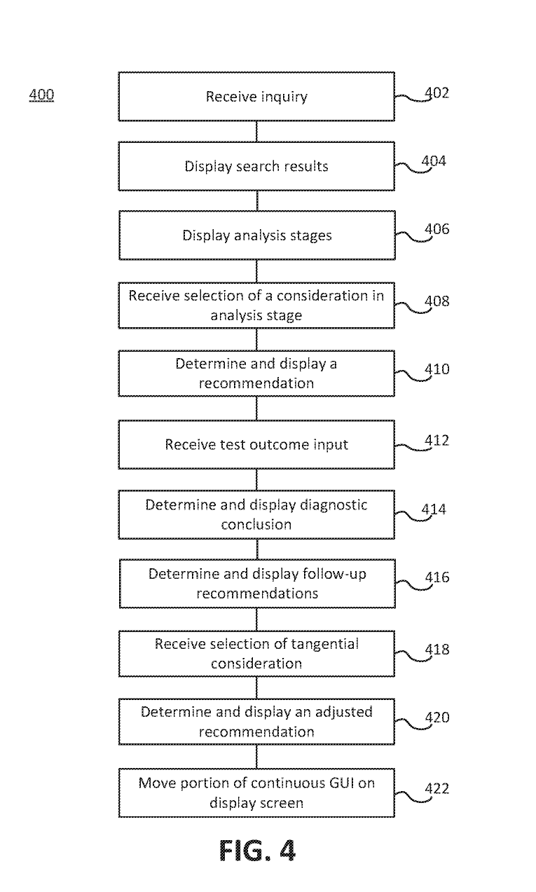

As an example of utilizing a continuous GUI 146, method 400 for analyzing information using continuous GUI 146 is depicted in FIG. 4. With combined reference to FIGS. 1, 2A-2C, and 3A-3C, a user of system 100 may search for a medical topic (e.g., hepatitis) by typing an inquiry into search feature 205. Processor 110 may receive the inquiry (step 402), access medical information 122 in memory device 120, and search for results matching the inquiry (e.g., information relating to hepatitis). Processor 110 may display search results 235 (step 404) for the medical topic 207 on display screen 132. The user may select for which of search results 235 the user would like receive further information, and processor 110 may receive the selection and retrieve the information associated with the selected search result 235 from memory device 120. In response, processor 110 may display a summary screen 250 with information options 254, 257, and/or 259, which the user may select to view the respective information. For example, in response to the user selecting a summary information option 254, summary information 255 for the selected search result may be displayed on display screen 132.

In various embodiments, in response to the user selecting an instruction information option 257, processor 110 may display a continuous GUI 146 on display screen 132. Continuous GUI 146 may comprise and display various analysis stages (step 406), which a user may work through in order to receive a recommendation regarding how to proceed with a patient (e.g., diagnostic testing and/or treatment). Each analysis stage may comprise information and/or considerations which the user will review, consider, and select in order to allow processor 110 to generate additional analysis stages and information. For example, continuous GUI 146 may begin with view 300A in viewing column 399A, displaying a first analysis stage 352 comprising background considerations 305. Background considerations 305 may be information relating to a patient occurring before the present medical inquiry (e.g., occurrences heightening the risk of hepatitis C contraction, previous conditions, previous test or examination results, patient characteristics, or the like). The user may select one or more of the background considerations 305 to indicate patient traits or factors (e.g., by selecting selectors 349A), and processor 110 may receive such selections (step 408). The background considerations and/or the selections of a background consideration(s) may be stored in memory device 120 for future use by processor 110. Based on the selected background considerations, processor may determine and display a recommendation(s) (step 410), which may be a test recommendation 308. In the example shown in FIG. 3A, test recommendation 308 may be determined because one or more background considerations had been selected, triggering test recommendation 308 (e.g., to test for the Hepatitis C antibody). Test recommendation 308 may be displayed (step 410) proximate or adjacent to (e.g., below) background considerations 305, making it easy and efficient for a user to simply scroll a short distance on continuous GUI 146 to view test recommendation 308, or to review background considerations 305 once again.

In various embodiments, a continue button 302 may be provided on continuous GUI 146 after one or more analysis stages (e.g., first analysis stage 352), which the user may select to instruct processor 110 to determine and/or display a subsequent analysis stage or subsequent information, such as a second analysis stage 354, which may include test recommendation 308. Second analysis stage 354 may be displayed on continuous GUI 146 proximate or adjacent to (e.g., below) test recommendation 308 and/or background considerations 305 (i.e., viewing column 399B may be a continuation of column 399A). The subsequent analysis stage may comprise an input device 150, allowing the user to input the outcome of the recommendation. For example, test outcome input 310 allows the user to indicate to processor 110 whether the test performed as recommended by test recommendation 308 was positive or negative (e.g., by selecting “positive” or “negative” in test outcome input 310), and processor 110 receives test outcome input 310 (step 412). A test outcome input may also comprise historical test or examination results about the patient input into system 100. Test outcome input 310 may cause processor 110 to determine and display one or more follow-up analysis stage 356, such as an additional test recommendation 312 to test for Hepatitis C RNA in response to a positive Hepatitis C antibody test outcome input 310. Follow-up analysis stage 356 may be displayed proximate or adjacent to (e.g., below) test outcome input 310 so the user can easily continue to see the analysis progression of determining if a patient has Hepatitis C (e.g., by scrolling on a display screen 132). In response to a positive test outcome input 314, processor 110 may determine and display (proximate or adjacent to, e.g., below, test outcome inputs 310 and/or 314) a diagnostic conclusion 318 (step 414) (which in FIG. 3A shows a Hepatitis C diagnosis in response to the positive test outcome inputs 310 and 314).

In response to the determination and display of diagnostic conclusion 318, processor 110 may determine and display one or more follow-up recommendations 322, 324, 326 (step 416) proximate to (e.g., below) diagnostic conclusion 318 on continuous GUI 146. For example, follow-up recommendation 322 recommends working up concomitant causes of liver disease to Hepatitis C, follow-up recommendation 324 recommends various actions in preparation for treatment, and follow-up recommendation 326 advises of likely effect of treatment.

In various embodiments, processor 110 may determine and display tangential considerations related to analysis of a situation and/or patient on continuous GUI 146 proximate or adjacent to a previously presented recommendation (e.g., test recommendation 308, and/or follow-up recommendations 322, 324, 326). The tangential consideration(s) may be comprised in an analysis stage 358. For example, tangential consideration may be something not medically related to diagnosis or treatment of a patient, but is relevant to maximize the efficiency and/or effectiveness of the process, such as tangential consideration 328 which inquires about the patient's financial means. For example, if the patient cannot afford the best testing and/or treatment, processor 110 may determine the most cost-efficient manner in which to utilize the patient's resources to maximize the health benefit. This may be completed by processor 110 determining and recommending first those processes and/or treatments are most important to a patient's wellbeing, which may be marked with a ranking marker by processor 110. Processor 110 may receive a selection of tangential consideration 328 (step 418), and may determine and display an adjusted recommendation (step 420) by adjusting a previous or subsequent recommendation based on tangential consideration 328. For example, if cost is a factor, as indicated by a selection on tangential consideration 328, processor 110 may provide an adjusted recommendation (e.g., a test recommendation or treatment recommendation) simplifying a previous or subsequent recommendation to lower the total cost of the analysis processes and/or treatment.

Continuous GUI 146, to which processor 110 continues to add by displaying additional information and/or analysis stages (e.g., below previously displayed items, such as information and analysis stages), may extend for as far as needed on display screen 132 for processor 110 to receive all relevant information from user to make the appropriate recommendation. For example, FIG. 3B, in accordance with various embodiments, shows another view 300B of continuous GUI 146, starting with viewing column 399C, which may be displayed below as a continuation of (e.g., below) viewing column 399B (depicted in FIG. 3A) on display screen 132. Processor 110 may display in viewing column 399C another analysis stage 362 inquiring about high mortality/morbidity features (i.e., additional considerations) a patient may have. Analysis stage 362 may be displayed proximate or adjacent to (e.g., below) tangential consideration 328, or any other related or relevant consideration. In response to receiving a selection and/or a command to continue (e.g., by a user selecting selectors 349B), processor 110 may determine and display a final recommendation 332. In this example, final recommendation 332 advises to initiate treatment. Final recommendation 332 is based on all previous information gathered by processor 110 through the analysis stages, which all can be viewed on continuous GUI 146 by scrolling to find the desired information. In various embodiments, final recommendation 332, or the last analysis stage of an instruction (in this example, final recommendation 332 is the end of the screening and diagnosis instruction), there may be an advance button 306. In response to a user selecting advance button 306 at the end of an instruction, processor 110 may begin a subsequent instruction related to the previous instruction (in this example, the subsequent instruction would be Hepatitis C treatment portion 340 displayed in viewing column 399D, which may be displayed below as a continuation of (e.g., below) viewing column 399C).

In various embodiments, all information and/or analysis stages completed and displayed on continuous GUI 146 may not be visible at once on display screen 132. Therefore, a user may scroll to find desired information, which remains displayed in the logical order by which the user and processor 110 analyzed the subject situation and/or patient. For example, to review previous analysis stages, a user may use a mouse or their finger on a touchscreen to scroll back to the previous information. For example, on a mobile device, all information and analysis stages may be displayed linearly, with the newest information displayed below previous information (or vice versa). In various embodiments, a heading, such as 301A or 301B, may remain at the top of display screen 132 while the user is scrolling through information and/or analysis stages. A user may scroll up on the display screen of the mobile device to view previous analysis stages and information where continuous GUI 146 displayed the desired information. Processor 110 may receive the user's command to move to a different portion of continuous GUI 146 such that different information in continuous GUI 146 is visible on display screen 132, and may move continuous GUI 146 to display the desired portion on display screen 132 (step 422). Such a display on continuous GUI 146 allows the user (e.g., a medical professional) to easily navigate and reference-back through the various analysis stages and information to quickly make an informed decision for a patient (and/or make necessary changes/revisions in previous analysis stages).

With continued reference to FIGS. 1, 2A, and 3B, in response to processor 110 determining and displaying final recommendation 332 to begin treatment, processor 110 may begin Hepatitis C treatment portion 340 on continuous GUI 146. In various embodiments, hepatitis C treatment portion 340 may begin in response to advance button 306 being selected. The analysis stages for treatment portion 340 may be displayed proximate or adjacent to (e.g., below) final recommendation 332, or any other relevant or related information or analysis stages. Analysis stage 372 may comprise a condition selection list so a user can indicate to processor 110 which condition will be treated. In response to a condition selection, processor 110 may determine and display background considerations which need to be acknowledged, such as those listed in analysis stage 374 (whether the patient has been treated for the condition before). The background considerations may be displayed proximate or adjacent to the condition list on continuous GUI 146. Processor 110 may continue to determine and display additional background considerations 335 (such as those displayed in viewing column 399E in FIG. 3C, which may be proximate/adjacent to (e.g., below), or a continuation of, viewing column 399B), which may inquire as to the patient's genotype and current afflictions (e.g., cirrhosis). As depicted in FIG. 3C, background considerations 335 are broken up into various analysis stages: analysis stage 376 for the patient's trait(s) (e.g., genotype), analysis stage 378 for a sub-trait (e.g., genotype subtype), and analysis stage 384 for indicating an additional trait (e.g., whether the patient has cirrhosis). In response to the inputs received by processor 110 from the analysis stages, including the condition selection list and background considerations about the patient, processor may determine and display a treatment recommendation 380.

In various embodiments, with continued reference to FIGS. 1 and 3A-3C, one or more selection options in each analysis stage may comprise an information button 303 (an example of an information tool 148). A user may select information button 303, processor 110 will receive the selection of information button 303, and processor 110 will display information associated with the consideration or category associated with the selected information button 303. For example, if a user selects information button 303 in analysis stage 376 for “Genotype 1,” information about genotype 1 will be displayed on display screen 132 such as the percentage of the population who has genotype 1, treatment effectiveness for genotype 1, or the like. Another example of an information tool 148 may be a comment button 304 comprised in one or more selection options in each analysis stage. A user may select comment button 304, processor 110 will receive the selection of comment button 304, and processor 110 may display a screen through which the user may enter a comment relating to the consideration, category, or topic for viewing by other future users. Previous comments left by previous users may be accessed by a current user selecting comment button 304 and/or information button 303.

Continuous GUI 146, as discussed in relation to FIGS. 2A-4, is a step-by-step method that a medical professional may use to analyze a situation and/or patient. Every step in the analysis may be utilized, and therefore, continuous GUI 146 may be implemented for medical professionals unfamiliar with certain subject matter, such that continuous GUI 146 may guide them through the entire analysis. To further increase efficiency of system 100 providing medical information and guidance to users, however, in various embodiments, GUI 140 may take the form of a GUI inquiry map 144.

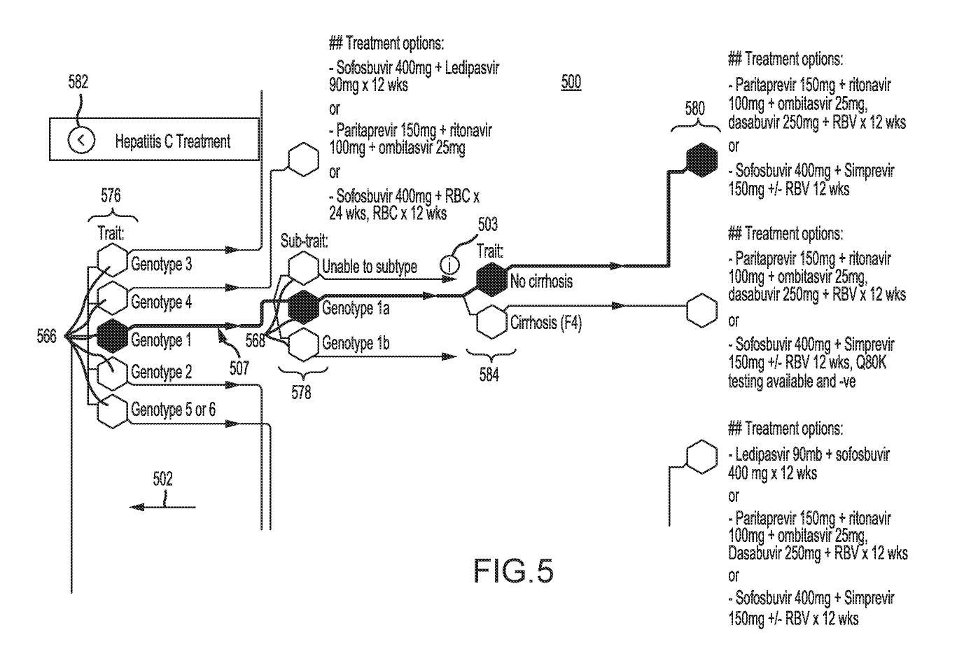

With reference to FIG. 5, in accordance with various embodiments, GUI inquiry map 500 (an example of GUI inquiry map 144 in FIG. 1) may be utilized by a user who has knowledge of a subject and wishes to quickly navigate through various analysis stages. For example, with additional reference to FIG. 1, a user may enter a search inquiry in search feature 142, and may elect to view and use GUI inquiry map 500 associated with the desired search result. GUI inquiry map 500, in various embodiments, may comprise and display analysis stages, wherein each analysis stage comprises at least one node for a user to select to indicate to process information about the situation and/or patient. For example, GUI inquiry map 500 displays analysis stage 576 with nodes 566, from which the user may select a trait (e.g., the genotype of Hepatitis C) that the patient has. In response to selecting “Genotype 1” from nodes 566, processor 110 may determine and display a subsequent analysis stage 578 comprising nodes 568 for selecting a sub-trait (e.g., the subtype genotype) (or, in various embodiments, subsequent analysis stage 578 may already be on GUI inquiry map 500, and GUI inquiry map 500 may navigate to subsequent analysis stage 578 for better view on display screen 132). Finally, in the illustrative example of GUI inquiry map 500, processor may determine and display analysis stage 584 for indicating whether the patient has an additional or alternative trait (e.g., cirrhosis). Based on the selections in the analysis stages in GUI inquiry map 500, processor 110 may determine and display a recommendation (e.g., a treatment recommendation 580, a recommendation for a diagnostic test, or the like) associated with each trait and/or sub-trait selected.

As can be seen comparing FIGS. 3C and 4, analysis stages 576, 578, and 584 display the same selection options as analysis stages 376, 378, and 384 of continuous GUI 146, respectively. In various embodiments, GUI inquiry map 500 may provide an information button 503 (similar to information button 303) with each node, which, in response to being selected, may cause processor 110 to display information relating to the associated node on display screen 132. In various embodiments, GUI inquiry map 500 may not provide an information button 503 with each node, because users utilizing GUI inquiry map 500 presumably may not need additional information, and are using GUI inquiry map 500 because they may already have the knowledge to select the appropriate node. In various embodiments, GUI inquiry map 500 may provide a comment button (similar to comment button 304 depicted in FIG. 3A), which a user may select to add a comment and/or access previous comments left by other users of system 100.

In various embodiments, related nodes between analysis stages in GUI inquiry map 500 are linked by a path, and the selected path between selected nodes may be illuminated or otherwise marked. That way, should a user wish to navigate forward or backward (direction 502) on GUI inquiry map 500, the user cold follow the illuminated path. The entire GUI inquiry map 500 may not be visible on display screen 132 at once, so only a portion of GUI inquiry map 500 may be displayed at a given time. To navigate through GUI inquiry map 500, user may command processor to move a different portion of GUI inquiry map 500 to be displayed on display screen 132 by moving a mouse or dragging a finger or soft pen on a touch screen. Similar to continuous GUI, with example views shown in FIGS. 3A-3C, GUI inquiry map 500 allows a user to easily see the steps in an analysis of a situation and/or patient, simply by moving GUI inquiry map 500 to display different portions on display screen 132.

Additionally, users may skip certain analysis stages that they already know using GUI inquiry map 500. For example, a user may simply select the node 568 for a sub-trait (e.g., “Genotype 1a”) in analysis stage 578, and the associated trait (e.g., “Genotype 1”) node 566 in analysis stage 576 will automatically be selected. This allows medical professionals who are familiar with the subject matter at hand to skip those aspects of the analysis that are obvious or known, and can focus on more pertinent or esoteric information needed to complete the analysis. Also, if a user comes across an analysis stage or node with which they are not familiar, and needs more information to make a node selection in GUI inquiry map 500, an information button 503 may be provided in one or more analysis stages. Selecting the information button 503 may cause processor 110 to display information on the topic associated with information button 503 selected. For example, similar to selecting information button 303 in continuous GUI 146, if a user selects information button 503 in analysis stage 578, information about analysis stage 578 (e.g., sub-traits) will be displayed on display screen 132. The user may receive the same information by selecting information button 503 on GUI inquiry map 500 as the user would have received by selected an information button 303 for the same topic and/or analysis stage in continuous GUI 146.

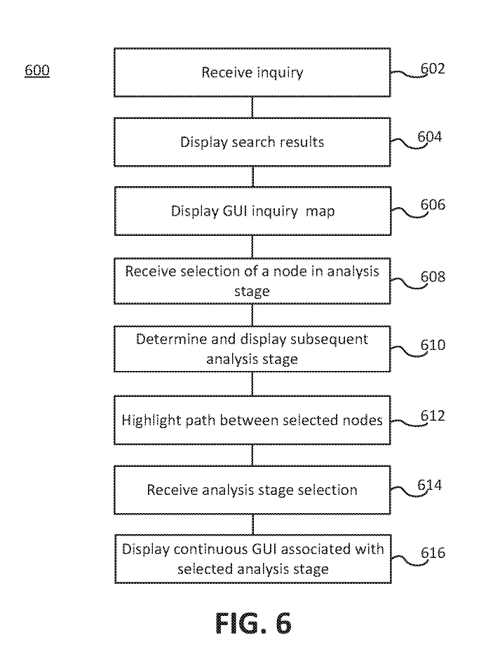

FIG. 6 depicts a method 600 for analyzing information using a GUI inquiry map, such as GUI inquiry map 500, of system 100 (in FIG. 1), in accordance with various embodiments. With combined reference to FIGS. 1, 2A, 4, and 6, processor 110 may receive an inquiry from a search feature 205 (step 602) and display search results 235 associated with the inquiry (step 604). The user may select which of search results 235 the user would like to pursue, and processor 110 may receive the selection and retrieve the information associated with the selected search result from memory device 120. Processor 110 may display information options 254, 257, and/or 259, which the user may select to view the respective information. For example, in response to the user selecting a summary information option 254, summary information 255 for the selected search result may be displayed on display screen 132.

In various embodiments, in response to a command from the user, processor 110 may display GUI inquiry map 500 (step 606) related to the selected search result. GUI inquiry map 500 may comprise at least one analysis stage (e.g., analysis stage(s) 576, 578, and/or 584). Each analysis stage may comprise one or more nodes (e.g., nodes 566 and/or 568), wherein related nodes between analysis stages are linked by a logical path. Processor 110 may receive a selection of a node in a first analysis stage (step 608), for example, as depicted in FIG. 6, selection of node 566 for a trait (e.g., “Genotype 1”) in analysis stage 576. In response, processor 110 may determine and display a subsequent analysis stage (step 610) (e.g., analysis stage 578). In various embodiments, related analysis stages may be determined and displayed on GUI inquiry map 500 prior to a selection of a node. In various embodiments, upon receiving selections of a node in each of two analysis stages, processor 110 may highlight the linked path between the selected nodes (step 612), such as highlighted path 507 between analysis stages 576 and 578.

In various embodiments, as discussed herein, a user may wish to skip ahead to an analysis stage that is not at the beginning of the logical order of analysis stages. For example, the user may wish to skip to analysis stage 578, because the user already knows about analysis stage 576 and those that may precede analysis stage 576. Therefore, the user may select analysis stage 578 of interest, and processor 110 may receive the selection (step 614). In response, processor 110 may fill in related nodes in analysis stages preceding the analysis stage selected. For example, if node 568 for a sub-trait (e.g., “Genotype 1a”) is selected in analysis stage 578, processor 110 may automatically highlight or fill in node 566 for the associated trait (e.g., “Genotype 1”) in analysis stage 576.

In various embodiments, the user may select analysis stage 578, and then select information button 503. In response, processor 110 may display information associated with the analysis stage and/or topic with which information button 503 is associated. In various embodiments, the user may select a node and/or a back button 582. In response, processor 110 may navigate the user to the continuous GUI view associated with the analysis stage 578 (step 616) displayed on display screen 132 and/or associated with the node, which, following the previous example, would be view 300C with analysis stage 378 comprising the selected sub-trait (e.g., “Genotype 1a”), depicted in FIG. 3C. In doing so, the user will have access to more in-depth information through information button 303 or other information tools 148. If the user is finished with the more in-depth information through continuous GUI 146, the user may navigate back to GUI inquiry map 500.

In various embodiments, additional information tools 148 may be available through continuous GUI 146 such as available medical trials for a patient having the correct criteria (determined through selections of considerations in continuous GUI 146 and/or nodes in GUI inquiry map 500). Medical trials displayed by processor 110 may account for tangential considerations such as financial reward and/or geographic location. By selecting a medical trial, processor 110 may display the information for the medical trial such as the type of study, measured outcomes, participant requirements, or the like. Another information tool 148 may be a discussion board on which users may post questions and answers, and/or view relevant articles relating to topics of interest. Yet another information tool 148 may be a surgery risk calculator, which takes into account risk considerations (e.g., type of surgery, age of patient, ejection fraction, hemodynamic state, or the like). Based on these considerations, which are input by the user based on the patient characteristics, processor 110 determines a predicted mortality risk, which is the percent chance that the patient may not survive the surgery. The mortality risk is calculated by adding the associated risk points of each risk consideration to generate a total risk score, which in turn is converted into a predicted mortality risk based on a relational database containing the risk score to predicted mortality risk numbers. It should be understood that the predicted mortality risk may be displayed in a percent chance that the patient will survive, or any other suitable rating or measure.

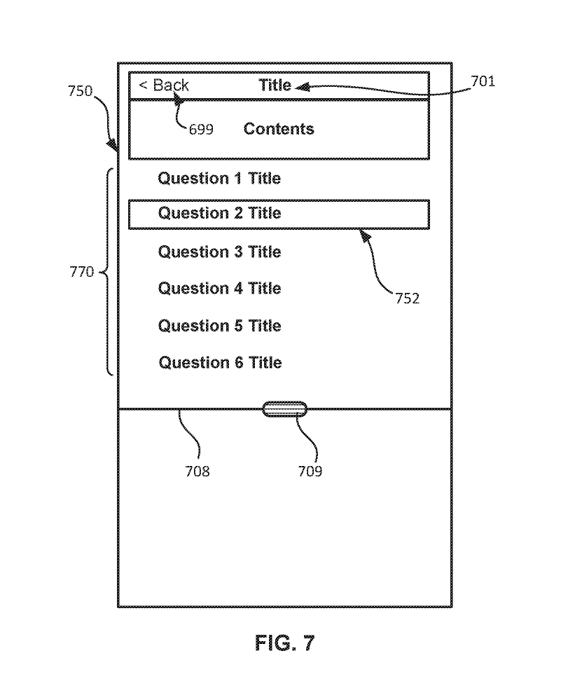

With reference to FIG. 1, to further increase efficiency of system 100 providing medical information and guidance to users, in various embodiments, GUI 140 may take the form of a windowed GUI 152. Windowed GUI 152 may take the form of a series of windows, at least some of which may be collapsible and expandable, superimposed or stacked, or otherwise disposed and arranged to display information allowing a user of system 100 to easily navigate between windows and/or displayed information. Exemplary views of windows in windowed GUI 152 are depicted in FIGS. 7-9.

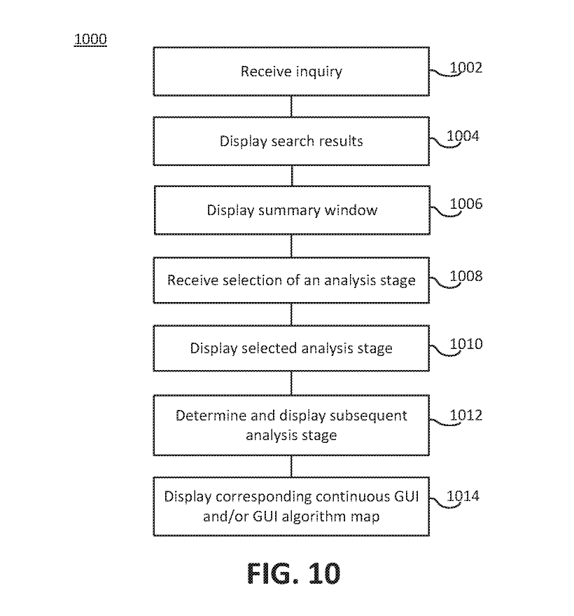

With combined reference to FIGS. 1 and 7-10, in various embodiments, a method 1000 for accessing information using a windowed GUI of a medical evaluation system 100 may comprise receiving an inquiry (step 1002) from a user (e.g., for a medical topic, such as hepatitis, the example used in relation to methods 400 and 600 discussed herein). Processor 110 may display the search results (step 1004), such that the user may select one (such as those displayed in FIG. 2B). In response to the user selecting a search result (e.g., a medical topic), processor 110 may display a summary window 750 (step 1006) for the selected topic. Summary window 750 may comprise a heading 701, which may display the title or name of the topic selected by the user. Windows in windowed GUI 152 may further comprise “back” buttons 699, which, in response to being selected, may cause processor 110 to display the previous window and/or screen of windowed GUI 152 on display screen 132.

Summary window 750 may display a number of analysis stages 770. Analysis stages 770 may comprise questions, inquiries, or topics to be explored (e.g., Questions 1-6), which may go through the diagnosis and/or treatment of a disease (e.g., the medical topic). In various embodiments, analysis stages 770 may be similar and/or correspond to analysis stages comprised in continuous GUI 146 shown in viewing columns 399A-399E in FIGS. 3A-3C, and/or may be similar and/or correspond to analysis stages in GUI algorithm map 144 shown in FIG. 5. For example, Question 2 of analysis stages 770 may comprise the title “Trait,” and may correspond to analysis stages 376 in continuous GUI 146 and/or analysis stage 576 in GUI algorithm map 144.

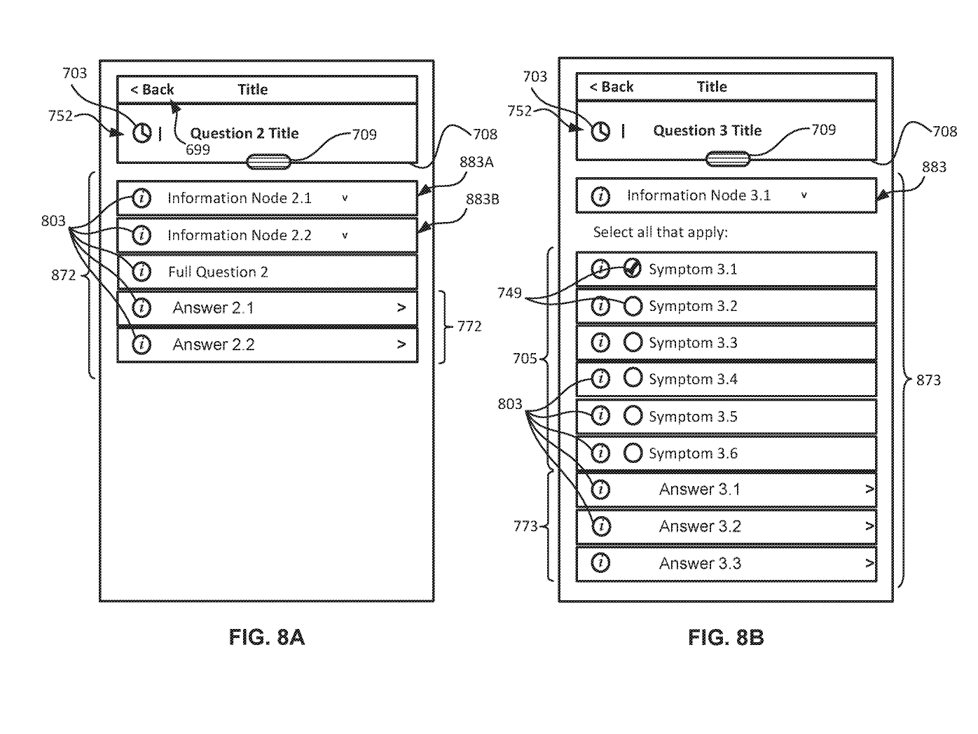

In various embodiments, as similarly discussed herein regarding use of GUI algorithm map 144, a user may wish to skip ahead to an analysis stage that is not at the beginning of the logical order of analysis stages. For example, the user may wish to skip to Question 2 of analysis stages 770 displayed in windowed GUI 152, because the user already knows about Question 1 of analysis stages 770 (or any other preceding analysis stages 770). Therefore, the user may select Question 2 of analysis stages 770, and processor 110 may receive the selection (step 1008). In response (or prior to selection), a view frame 752 may be disposed around Question 2 of analysis stages 770. Processor 110 may display on display screen 132 a window for the selected analysis stage 770 (step 1010) (e.g., a Question 2 window 872) comprising a number of items associated with the selected analysis stage 770 (e.g., information, text, images, selectors, and/or the like). In order to do so, in various embodiments, summary window 750 may be retracted (e.g., moved upward on display screen 132, such that bottom boundary 708 of summary window 750 is moved upward) to show items related to Question 2. Retracted summary window 750 may assume the configuration of only displaying the selected analysis stage 770, such as “Question 2” within view frame 752. Question 2 window 872, for example, may comprise and display information nodes 883A and 883B, the Full Question 2, and/or answers 772 to Full Question 2, shown in FIG. 8A. Information nodes 883A and 883B may be similar to information buttons 303 in continuous GUI 146 and GUI algorithm map 146, in that, in response to being selected, information nodes 883A and 883B may expand within Question 2 window 872 and display information associated with Question 2. Similarly, in various embodiments, information buttons 803 associated with one or more items comprised in a window (e.g., the items comprised in Question 2 window 872), may expand and display information associated with the item associated with the respective information button 803. In various embodiments, in response to a user selecting an information button 803, processor 110 may display a separate information window displaying information associated with the item associated with the respective information button 803.

In response to an answer 772 being selected by a user, processor 110 may receive the selection and automatically determine and display a subsequent analysis stage 770 (step 1012). For example, a user may select one of answers 772, and processor 110 may determine that the next appropriate analysis stage 770 is the next sequential analysis stage 770 (e.g., Question 3 of analysis stages 770 after Question 2). In various embodiments, the next appropriate analysis stage 770 may not be the next sequential analysis stage 770 based on the answer 772 selected. For example, processor 110 may determine and display a Question 4 window in response to a certain answer selection in Question 2 window 872.

In various embodiments, a user may be able to jump to any desired analysis stage 770 within the topic shown in heading 701. That is, if the user realizes that the selected analysis stage 770 is not that which was desired, and/or the user wants to skip subsequent analysis stages 770 after reviewing and/or answering an analysis stage 770, the user may navigate back to summary window 750 and select another analysis stage 770 (similar to step 1008). To do so, the user may select summary view button 709, which may expand summary window 750 onto display screen 132 over the items for the previously displayed analysis stage 770 (e.g., summary window 750 may be a drop-down menu). For example, the user viewing Question 2 window 872 may select summary view button 709, which may cause summary window 750 to expand (e.g., move downward on display screen 132, such that bottom boundary 708 of summary window 750 is moved downward to display analysis stages 770). The user may then select another analysis stage 770 to be displayed, and summary window 570 may retract in response. While summary window 750 is expanded as a result of summary view button 709 selection, in response to the user wishing to see the information displayed previously (before expanding summary window 750 by selecting summary view button 709), the user may select summary view button 709 while summary window 750 is expanded. In response, summary screen may retract, and the previous window may be displayed (e.g., Question 2 window 872).

In response to processor 110 determining that Question 3 is the appropriate subsequent analysis stage 770 in response to the user selecting at least one of answers 772, or in response to the user selecting Question 3 on summary window 750, processor 110 may display a Question 3 window 873, shown in FIG. 8B. Similar to Question 2 window 872, Question 3 window 873 may comprise items associated with Question 3, such as information node 883 (which may be similar to information nodes 882A,B), answers 773 to Question 3, and/or selectable factors or traits 705 (e.g., symptoms). For example, Question 3 may ask the user to describe a patient's condition, which the user may do by selecting selectors 749 to indicate the symptoms (traits 705) exhibited by the patient. Similar to the process described in relation to Question 2 window 872, a user may answer Question 3 on Question 3 window 873 by selecting one of answers 773, and processor 110 may determine and display a subsequent analysis stage 770.

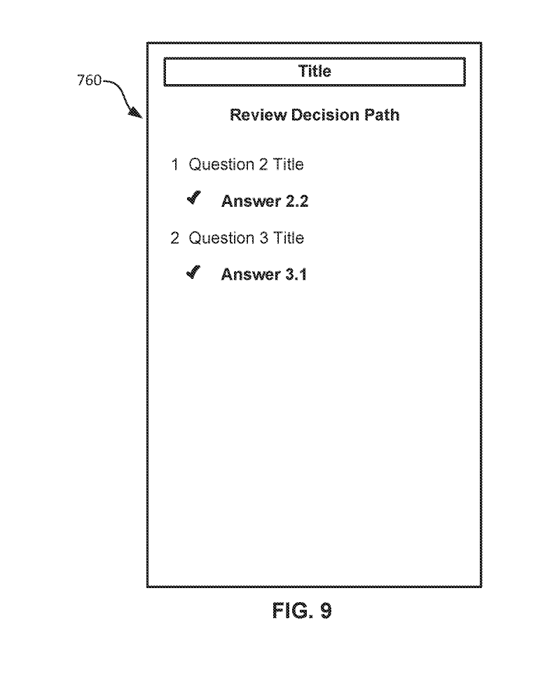

A user may navigate windowed GUI 152 until desired information is reached and displayed on display screen 132, such as a diagnosis or treatment recommendation. At any point, a user may search for a different medical topic (having a different algorithm than the current medical topic), search for a topic within the current medical topic (within the same algorithm), jump to another analysis stage 770 within a topic, and/or navigate back to a window comprising information that was previously viewed. For example, a user may select a query history button 703, which may be displayed on at least one window in the windows associated with an analysis stage or topic, and processor 110 will receive the user's selection of query history button 703 and display a history window 760, depicted in FIG. 9. History window 760 may display previous analysis stages 770 the user has selected and/or viewed, and inputs the user has made in respective analysis stages 770 (e.g., answers selected). For example, as shown in FIG. 9, while viewing Question 2 window 872, user may have selected Answer 2.2 of answers 772, and while viewing Question 3 window 873, user may have selected Answer 3.1 of answers 773. The user may navigate back to a previous analysis stage 770 by selecting it, or back to summary window 750 or another window my selecting a corresponding button to command processor 110 to do so.

Similarly, in various embodiments, a user may navigate between analysis stages in a topic (e.g., analysis stages 770 in a topic shown in heading 701) by swiping back and forth (e.g., left or right, or up and down) on display screen 132, where display screen 132 is a touch screen. “Swiping” may include a user touching a finger to display screen 132 and then moving it in a certain direction (e.g., left, right, up, or down). Processor 110 may detect the user's swipe and display an analysis stage 770 corresponding to the detected swipe. For example, swiping in a rightward (or upward) direction may cause processor 110 to display a display that was previously viewed (e.g., the immediately previous view) or a previous analysis stage 770, and swiping in a leftward (or downward) direction may cause processor 110 to display the next sequential analysis stage 770 or other related screen, or vice versa.

In various embodiments, a user may command processor 110 to navigate from windowed GUI 152 to the associated analysis stage in continuous GUI 146 and/or GUI algorithm map 144. For example, to see the GUI algorithm map 144 and/or continuous GUI 146 corresponding to the windowed GUI 152 for the selected topic, a user may select a button to command processor 110 to display the corresponding GUI algorithm map (e.g., GUI algorithm map 500) and/or continuous GUI (step 1014) and the corresponding analysis stages comprised therein. In response, processor 110 may make such a display, offering the user a different view of information.

While the systems and methods herein are discussed in relation to medical evaluation (e.g., diagnosis and/or treatment), it should be understood that the concepts and graphical user interface displays described herein may be utilized in any industry requiring ready access to information and multi-step evaluations.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, JAVA®, JAVASCRIPT, VBScript, Macromedia Cold Fusion, COBOL, MICROSOFT® Active Server Pages, assembly, PERL, PHP, awk, Python, Visual Basic, SQL Stored Procedures, PL/SQL, any UNIX shell script, and extensible markup language (XML) with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JAVASCRIPT, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “JAVA® Cryptography” by Jonathan Knudson, published by O'Reilly & Associates (1998); (3) “Cryptography & Network Security: Principles & Practice” by William Stallings, published by Prentice Hall; all of which are hereby incorporated by reference.

As will be appreciated by one of ordinary skill in the art, the system may be embodied as a customization of an existing system, an add-on product, a processing apparatus executing upgraded software, a stand alone system, a distributed system, a method, a data processing system, a device for data processing, and/or a computer program product. Accordingly, any portion of the system or a module may take the form of a processing apparatus executing code, an internet based embodiment, an entirely hardware embodiment, or an embodiment combining aspects of the internet, software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user WINDOWS®, webpages, websites, web forms, prompts, etc. Practitioners will appreciate that the illustrated steps described herein may comprise in any number of configurations including the use of WINDOWS®, webpages, web forms, popup WINDOWS®, prompts and the like. It should be further appreciated that the multiple steps as illustrated and described may be combined into single webpages and/or WINDOWS® but have been expanded for the sake of simplicity. In other cases, steps illustrated and described as single process steps may be separated into multiple webpages and/or WINDOWS® but have been combined for simplicity.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Although the disclosure includes a method, it is contemplated that it may be embodied as computer program instructions on a tangible computer-readable carrier, such as a magnetic or optical memory or a magnetic or optical disk. All structural, chemical, and functional equivalents to the elements of the above-described various embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method, comprising:
receiving, by a processor, an inquiry in a search feature;
displaying, by the processor, search results on a display screen in response to the inquiry;
displaying, by the processor, at least one analysis stage on a continuous graphical user interface (GUI) on a display screen, wherein the at least one analysis stage comprises a plurality of considerations;
receiving, by the processor, a selection of a consideration of the plurality of considerations;
determining, by the processor, a recommendation based on the selection of the consideration; and
displaying, by the processor, the recommendation on the continuous GUI proximate to the plurality of considerations.

2. The method of claim 1, wherein the displaying the at least one analysis stage comprises displaying, by the processor, at least one background factor for selection relating to a patient on the continuous GUI on the display screen, wherein the receiving the selection comprises receiving, by the processor, at least one selection of the at least one background factor.

3. The method of claim 2, wherein the recommendation is a test recommendation, and wherein the method further comprises:
receiving, by the processor, a test outcome input associated with the test recommendation, wherein options for the test outcome input are displayed on the continuous GUI proximate to the test recommendation;
determining, by the processor, a diagnostic conclusion in response to the receiving the test outcome; and
displaying, by the processor, the diagnostic conclusion on the continuous GUI proximate to the diagnostic conclusion.

4. The method of claim 3, further comprising:
determining, by the processor, at least one of a follow-up recommendation or a treatment recommendation in response to the determining the diagnostic conclusion; and
displaying, by the processor, at least one of the follow-up recommendation or the treatment recommendation on the continuous GUI proximate to the diagnostic conclusion.

5. The method of claim 1, further comprising
receiving, by the processor, a tangential consideration related to the patient;
determining, by the processor, an adjusted recommendation in response to receiving the tangential consideration; and
displaying, by the processor, the adjusted recommendation on the continuous GUI proximate to the tangential consideration.

6. The method of claim 5, further comprising moving, by the processor, a portion of the continuous GUI on the display screen in response to receiving, by the processor, a moving command, wherein at least a portion of the continuous GUI is displayed on the display screen.

7. The method of claim 5, further comprising:
displaying, by the processor, an information button on at least one of the at least one background factor, the test recommendation, the test outcome, the diagnostic conclusion, the follow-up recommendation, or the adjusted recommendation; and
displaying, by the processor, information on the continuous GUI in response to a selection of the information button.

8. The method of claim 5, further comprising displaying, by the processor, a treatment recommendation based on at least one of the diagnostic conclusion, the tangential consideration, or the at least one background factor, wherein the treatment recommendation is displayed on the continuous GUI proximate to the diagnostic conclusion.

9. The method of claim 4, further comprising:
determining, by the processor, trial options associated with the diagnostic conclusion based on at least one of the at least one background factor, a treatment recommendation, or a tangential consideration; and
displaying, by the processor, the trial options on the continuous GUI.

10. A method, comprising:
receiving, by a processor, an inquiry in a search bar;
displaying, by the processor, a graphical user interface (GUI) inquiry map comprising a plurality of analysis stages related to the inquiry on a display screen, wherein the plurality of analysis stages are displayed proximate to related analysis stages in a logical order, and wherein each analysis stage comprises at least one node linked to a node in an adjacent analysis stage;
receiving, by the processor, a first selection of a first node in a first analysis stage of the at least one background factor; and
displaying, by the processor, a second analysis stage related to the first selection of the first node proximate the first analysis stage.

11. The method of claim 10, further comprising:
highlighting, by the processor, the first node and a link between the first node and the second analysis stage in response to the receiving the first selection.

12. The method of claim 10, further comprising moving, by the processor, a portion of the GUI inquiry map on the display screen in response to receiving, by the processor, a moving command, wherein at least a portion of the GUI inquiry map is displayed on the display screen.

13. The method of claim 12, further comprising:
receiving, by the processor, an analysis stage selection of a selected analysis stage of the plurality of analysis stages, wherein the analysis stage selection allows a user to skip at least one preceding analysis stage the selected analysis stage in the logical order.

14. The method of claim 13, further comprising:
displaying, by the processor, a continuous GUI on the display screen comprising information associated with the analysis stage selection, wherein the continuous GUI comprises at least one of factors and considerations relating to a patient to aid in making a final recommendation;
receiving, by the processor, at least one selection of the at least one background factor; and
determining, by the processor, a recommendation based on the at least one selection.

15. The method of claim 13, further comprising highlighting, by the processor, the at least one preceding analysis stage skipped by the analysis stage selection of the selected analysis stage and at least one preceding link linking the selected analysis stage and the at least one preceding analysis stage.

16. A system comprising:
a processor; and
a tangible, non-transitory memory configured to communicate with the processor, the tangible, non-transitory memory having instructions stored thereon that, in response to execution by the processor, cause the processor to perform operations comprising:
receiving, by the processor, an inquiry in a search bar;
displaying, by the processor, a graphical user interface (GUI) inquiry map comprising a plurality of analysis stages related to the inquiry on a display screen, wherein the plurality of analysis stages are displayed proximate to related analysis stages in a logical order, and wherein each analysis stage comprises at least one node linked to a node in an adjacent analysis stage;
receiving, by the processor, a first selection of a first node in a first analysis stage of the at least one background factor; and
displaying, by the processor, a second analysis stage related to the first selection of the first node proximate the first analysis stage.

17. The system of claim 16, wherein the operations further comprise highlighting, by the processor, the first node and a link between the first node and the second analysis stage in response to the receiving the first selection.

18. The system of claim 16, wherein the operations further comprise moving, by the processor, a portion of the GUI inquiry map on the display screen in response to receiving, by the processor, a moving command, wherein at least a portion of the GUI inquiry map is displayed on the display screen.

19. The system of claim 18, wherein the operations further comprise receiving, by the processor, an analysis stage selection of a selected analysis stage of the plurality of analysis stages, wherein the analysis stage selection allows a user to skip at least one preceding analysis stage preceding the selected analysis stage in the logical order.

20. The system of claim 19, wherein the operations further comprise highlighting, by the processor, the at least one preceding analysis stage skipped by the analysis stage selection of the selected analysis stage and at least one preceding link linking the selected analysis stage and the at least one preceding analysis stage.

21. A system comprising:
a processor; and
a tangible, non-transitory memory configured to communicate with the processor, the tangible, non-transitory memory having instructions stored thereon that, in response to execution by the processor, cause the processor to perform operations comprising:
receiving, by the processor, an inquiry in a search bar;
displaying, by the processor, a windowed graphical user interface (GUI) comprising a collapsible summary window displaying a plurality of analysis stages related to the inquiry on a display screen in response to the summary window being in an expanded configuration, wherein the plurality of analysis stages are displayed proximate to related analysis stages in a logical order;
receiving, by the processor, a first selection of a first analysis stage of the plurality of analysis stages;
retracting, by the processor, the summary window; and
displaying, by the processor, a first analysis stage window associated with the first analysis stage.

22. The system of claim 21, wherein the operations further comprise:
receiving, by the processor, a selection of a summary view button;
expanding, by the processor, the summary window on the display screen over the first analysis stage window in response to the receiving the selection of the summary button.

23. The system of claim 21, wherein the operations further comprise:
receiving, by the processor, a selection of a query history button; and
displaying, by the processor, a history window displaying a user's activity history in the system and visited analysis stages.
